(19) United States
(12) Patent Application Publication
(10) Pub. No.: US 2023/0290518 A1
(43) Pub. Date: Sep. 14, 2023

(54) System for Long-Term Care Mitigation Providing a Secured Multi-step Trigger Cycle, and Method Thereof

(71) Applicant: Swiss Reinsurance Company Ltd., Zurich (CH)

(72) Inventors: Matthew William SINGLETON, Rickmansworth Hertfordshire (GB); Alan MARTIN, Romney Marsh Kent (GB); Eva RICHARDSON, London (GB); Sean WHITE, Bredgar Kent (GB)

(73) Assignee: Swiss Reinsurance Company Ltd., Zürich (CH)

(21) Appl. No.: 18/319,025

(22) Filed: May 17, 2023

Related U.S. Application Data
(63) Continuation of application No. PCT/EP2021/054445, filed on Feb. 23, 2021.

(30) Foreign Application Priority Data
Feb. 23, 2021 (WO) PCT/EP2021/054445

(51) Int. Cl. G16H 50/30; G06Q 40/06

(52) U.S. Cl. CPC G16H 50/30; G06Q 40/06

(57) ABSTRACT

Proposed is an automated system providing an automatable long-term care risk mitigation structure associated with immediate care needs of risk-exposed individuals in case of becoming a long-term care-receiver. The system provides an automated multi-step trigger and detection cycle based on sensory measuring data for risk-exposed individuals thereby mitigating possibly occurring long-term care needs by means of the automated system based on a detected sufficient state of disability, possibly predetermined up-front amount parameters and an assigned care equity ratio associated with an asset. The state of disability of the risk-exposed individual is measurable by a definable set of risk-exposed individual measuring parameters.

### CROSS-REFERENCE TO RELATED APPLICATIONS

This application is a continuation of and claims benefit under 35 U.S.C. § 120 to International Application No. PCT/EP2021/054445, filed Feb. 23, 2021, the entire contents of which are incorporated herein by reference.

### FIELD OF THE INVENTION

The present invention relates to automated and automatable care founding systems or care managing/planning systems, in particular to automated mortgage life/disability risk-transfer systems and systems for automated pricing, steering, and signaling mechanisms of such long-term care mitigation structures or risk-transfer structures. More particularly, the generally relates to systems and methods for the automation of absorption processes for long term care risk and automatic founding of long-term risk cover as the structuring of automated risk-transfers directly or indirectly associated with longevity risk or in connection with life risks covers. The inventions also generally relate to systems and methods for providing such credit support and safeguarding against longevity risk. The present invention further relates to systems and methods for structuring and providing credit support and fencing off longevity risk through investment arbitrage.

### BACKGROUND OF THE INVENTION

Long-term care (LTC) mitigation or risk-transfer is based on risk-transfer covers that provide support and hedges resources that help cover the cost of long-term care a person might need in the event of a chronic illness and unable to perform without substantial assistance at least some of the activities of daily living (bathing, continence, dressing, eating, toileting, transferring) or require substantial supervision due to a cognitive impairment, such as Alzheimer's disease. By helping to protect the person's assets, and giving the person choice and control over where he or she receives care, including in the person's own home, LTC risk-transfer helps a person and his or her family face the future with confidence.

In the prior art, there are various technical contributions to the insurance technology field of immediate and deferred care plan schemes, as Long Term Care (LTC) risk-transfer, mortgage life risk-transfer and disability risk-transfer systems. Immediate and deferred care plan schemes are long term risk-transfer covers which are designed to pay a regular, typically tax-free monetary amount (income) to the risk-exposed person's care provider to help meet the costs of his/her care. Deferred care plans for long term care pay out after a few months or years, instead of paying out benefits immediately, and are typically cheaper than immediate care plan schemes. There are at least two risk-transfer scheme, namely (i) Immediate Need Annuity (INA) and (ii) Lifetime mortgage (LTM—equity release): (i) INA is a type of annuity, also known as Immediate Care Plan or Care Fees Annuity, that pay out a guaranteed income for life to help cover the cost of the insured care fees in exchange for a one-off lump sum payment. The payments are typically set to pay a level benefit, or are index linked to raise over time. The advantages of the INA insurance scheme are that it has no time or cost limit. INA pays out, as long as it is needed by the insured. However, the one-time upfront fee, i.e. the product costs, are typically high, e.g. around 150,000 Euros (e.g. inter alia depending on the insured's level of income), and the insured cannot normally cancel or get the lump sum premium back, except during the statutory cooling-off period (e.g. 6 weeks: the insured normally need to pay an extra premium for some form of short-term mortality cover) after purchase; (ii) In the LTM scheme, a loan secured on the insured's estate (a mortgage loan) is made. Compounded interest is added to the capital throughout the term of the loan, which is then repaid by selling the property when the borrower (or borrowing couple) dies or moves out (perhaps into a care home). The borrower retains legal title to the home whilst living in it, and also retains the responsibilities and costs of ownership. The advantages of LTM are, inter alia, that it bears only a small amount of arrangement costs (e.g. 1,500 Euros). However, there are time (e.g. 8 years) and cost (e.g. 250,000 Euros) limits for the payout. Once the insured reaches one of the limits and is still alive, he/she will have to find other means to pay for the care. It is to be noted that LTMs are mainly sold for income/lump sum purchase purposes usually at a younger age. Thus, in certain cases, LTM is difficult to get as an instrument to fund care.

Over the last years, some of the disclosed risk-transfer systems tried to link their pooled resources to capital markets, thus exploring ways to diversify risk traditionally associated with exposure measures to fixed income, real estate, foreign exchange markets and commodities. As a result, the pooled financial resources are made available for use as alternative investment vehicles. One technical area has been the linkage of capital markets in the life risk-transfer industry. Life risk-transfer as an asset class is generally considered uncorrelated to the other segments of the financial markets, i.e., because mortality events measured to occur irrespective of the performance or condition of other economic metrics in the financial markets. Premium determination and allocation and life settlements have been specific technical areas in the life risk-transfer sector. Premium allocation as used herein is the allocation of premiums based on defining risk-transfer parameter settings, which are typically defined by policies. Moreover, life settlements as used herein are life risk-transfer policies sold by their owners to third parties in return for a lump sum payment. One technical alternative approach is the resource pooling in life risk-transfers through longevity arbitrage, which is being viewed by an increasing number of capital sources as an attractive alternative to traditional risk exposure. As a result, various longevity risk strategies and schemes exist. Traditionally, capital sources have primarily used the senior life settlement market to buy longevity exposure. Buying longevity exposure through the senior life settlement market, however, does not allow investors to effectively acquire the level of longevity assets necessary to achieve the desired risk diversification. In addition, most of these structures have major draw-backs under the technical requirement to be provided by an automated system.

Accordingly, it is desirable to provide automated systems and methods for safeguarding against longevity risk, as Long Term Care risks, without significant exposure to the shortcomings of the existing risk-transfer systems and methods. Additionally, the prior art risk-transfer systems invariably confronts chronically ill, physically or cognitively disabled, or frail elderly health care receivers with significant inadequacies of the processes of LTC risk-safeguarding, care management, planning and appropriate resource funding. First, many parts of the risk-transfer mechanisms are complicated, inconsistent and disconnected. Indeed, the only point of convergence and continuity in the current systems is the client. Patients and clients are often passed from system to system, and are often subjected to redundant assessments and evaluations, with little information passing from one risk-transfer resource pool to another risk-transfer resource pool. Second, the information clients are given from one system to another system is often non-transparent, incomplete, inaccurate, and in their actual consequences difficult to understand for a user. This results, inter alia, from no consistent, standardized assessments and no standardized structures. Third, because of short-term-stay structures and models that dominate in-patient health care, and because of short-term and intermittent care models in rehabilitation and home health care services, and because of large caseloads in community-based aging and disability services, clients in need of long-term care support services have difficulties to orientate themselves in the market of complex systems. Moreover, as is the case for most people, such care receivers cannot afford to buy long-term care risk-transfer policies, or are ineligible for coverage to begin with, or they cannot afford to pay for the care they need for as long as they need such long-term care. Fifth, and perhaps most importantly, there is no resource-planning structure that allows long-term care consumers to develop care-funding plans that will enable them to actually organize their own resources and/or acquire the resources they need to pay for the prevention-focused, long-term care that supports and promotes the reduction of unplanned hospitalization and emergency room visits among those most vulnerable to requiring unplanned in-patient care.

Finally, US 2017/0301032 A1 discloses an automated system for multi-pillar triggered risk-transfer intended to prolonge independent living under elderly illness occurrence. The system filters age-related parameters of elderly persons requesting to assign to the exposure-cover provided by the system by parameter values greater than a predefined age-threshold value. The system comprise a generated trigger-table with predefined searchable acute and/or chronic elderly illness parameters triggered in a patient dataflow pathway by a first trigger level for the occurrence of serious elderly illnesses, and in a second trigger level for the occurrence of an acute broken bone trauma, and in a third trigger level for long-term care indications, and in a fifth trigger level for assisted living indications, and the predefined parameters of a fifth-layer data structure providing measuring parameters for extended assistance indications, as defined by the trigger table. The system allows to be triggered multiple occurrences of acute and/or chronic elderly illnesses, and in consequence to dynamically adapt the cover due to the sequence of triggered acute and/or chronic elderly illnesses, which was not possible before.

In summary, there are significant physical, and emotional strains on a person and the person's family when he/she needs care, in particular long-term care. The financial complexities on top of this are most unwelcome. The options facing those persons are limited, and circumstances can often result in having to sell the family home against the person's care wishes. Thus, there should be a simple and automatable system benchmarked on its ability to automatically cover and to service financial care obligations of expectedly or unexpectedly arising long-term care needs, in particularly being designed to prevent persons with long-term care needs having to sell their home immediately and/or being forced to move out of their homes—perhaps ever. The system should work whether receiving care in the own home or in a specialist facility. There is an urgent need for transparent, technical solutions to the problems created by the present non-standardized process and prior art systems of long-term care funding and long-term care risk-transfer structures.

### SUMMARY OF THE INVENTION

It is an object of the invention to provide an automation based on a new long-term care risk mitigation system and structure covering care needs of risk-exposed individuals in case off or in the event of becoming a long-term care-receiver, thereby providing a secured multi-step trigger cycle for risk-exposed individuals. Therefore, a system enabled to cover immediate needs and also enabling possible future pre-pay-based structures. The novel system end method shall not only be able to cover and capture risks of the cost of long-term care, but also the risks of the occurring needs, in particular financial needs to cover possible treatments during the duration of the need, which cannot be handled by the traditional life or health risk-transfer systems and structures. Thus, the inventive system shall be able to provide a new risk-transfer landscape capable of handling the discussed risk-transfer gaps. Further, it is an object of the invention to provide a new event-driven and triggered system for automated optimization and adaption in signaling generation by dynamically triggering and/or monitoring new occurring individual measuring parameters associated with long-term care risks. The invention should specifically allow for mitigating the payment of care costs, using stable funding sources. Thus, the invention should further allow for providing the option to receive care in own home, or in a care home, at the will of the care receiver. It should further allow to remove the emotional burden and costs related to a rushed property sale in case of sudden long-term care needs. The invention should also allow to optimize tax benefit requirements when paying for care and for inheritance. The invention should also allow homeowners, in case of becoming a long-term care receiver, to retain, if possible, their asset until death/house sale, where e.g. their families can retain property when repaying the amount used. It should also allow families to benefit from any property appreciation. The invention should also allow to provide the option to rent out the home to help fund care and/or inheritance.

According to the present invention, these objects are achieved particularly through the features of the independent claims. In addition, further advantageous embodiments follow from the dependent claims and the description.

According to the present invention, the abovementioned objects for an automated system for long-term care risk mitigation associated with immediate care needs of risk-exposed individuals in case of becoming a long-term care-receiver, are particularly achieved by the described system, wherein the system provides a secured multi-step trigger cycle for risk-exposed individuals thereby mitigating possibly occurring long-term care needs by means of the automated system based on a sufficient state of disability, the state of disability of the risk-exposed individual being characterized by a definable set of risk-exposed individual measuring parameters, wherein individual measuring parameter values being captured and monitored by the automated system, in that the long-term care risk mitigation is triggered by means of an event-driven risk-exposed individual trigger providing the secured multi-step trigger cycle, the event-driven risk-exposed individual trigger comprising a staged multi-tier structure gradually triggering threshold values of a first and second set of individual measuring parameters in an autogenic dataflow pathway of a risk-exposed individual, wherein for each long-term care risk mitigation, long-term care mitigation data are held in a persistence storage of the system comprising at least one assigned relationship between an associated risk-exposed individual, an asset of the risk-exposed individual and a remedial measure ratio dependent on the asset of the risk-exposed individual, in that the staged multi-tier structure comprises a first trigger stage triggering on a sufficient state of disability of a selected risk-exposed individual by a first set of individual measuring parameter values captured in the dataflow pathway of the risk-exposed individual, the individual measuring parameter being segmentable in critical categories of individual measuring data at least comprising first trigger threshold parameter values providing a definable measure for individual's physical functional status parameters and/or current cognitive and/or sensory functional status parameters and/or prospective functional status parameters including physical and/or cognitive and/or sensory parameters and/or living environmental status parameters and/or long term care resource parameters, the trigger threshold parameter values triggered by means of monitored and measured values of the first set of individual measuring parameters in the autogenic dataflow pathway of a risk-exposed individual, in that upon detection of the sufficient state of disability based on the individual measuring parameters, the covering by means of remedial measures is activated by signal transmission to a risk-cover structure of the automated system and by transferring the remedial measures to the risk-exposed individual, in that the multi-tier trigger structure comprises a second stage trigger triggering a second set of individual measuring parameter values captured in the dataflow pathway of the risk-exposed individual based on second trigger threshold parameter values providing a definable measure for indicators for an occurred death of the risk-exposed individual and/or the sale of the real estate assigned to the risk-exposed individual, and the second threshold parameter values triggered by means of monitored and measured values of the measuring parameters in the dataflow pathway of the risk-exposed individual, and in that upon detection of the indicators for an occurred death of the risk-exposed individual and/or the sale of the asset assigned to the risk-exposed individual, the remedial measures transferred by the risk-cover structure of the system are returned to the system. Thus, the system can be based the two elements of the an upfront payment transfer (i.e. the risk-transfer or insurance element) and the repayment (i.e. the loan element which is the value of drawn-down home equity with no interest).

The inventive system has inter alia the advantage that it allows to provide an automatable, new risk mitigation and risk assessment structure for covering and mitigating long-term care risks in the context of life and health care technology. The system is able to complement the traditional risk-transfer cover in the life and health risk landscape, filling the gaps for long-term care risks. The system may be realized as a part of the classical risk-transfer multi-step trigger cycle. The invention further has the advantage to specifically allow for mitigating the payment of care costs, using stable funding sources, namely using an illiquid asset as the care-receivers residential property. The invention further allows for providing the option to receive care in own home, or in a care home, at the will of the care receiver. It further allows to remove the emotional burden and costs related to a rushed property sale in case of sudden long-term care needs. The invention also allows to optimize tax benefit requirements when paying for care and for inheritance. The invention also allows homeowners, in case of becoming a long-term care receiver, to retain their asset until death/house sale, where e.g. their families can retain property when repaying the amount used. The invention has the further advantage to allow families to benefit from any property appreciation, and to allow to provide the option to rent out the home to help fund care and/or inheritance. Upon reaching a sufficient state of disability, the potential long-term care receiver as annuitant pays an up-front longevity premium. The reaching of a sufficient state of disability typically is connected with reaching a certain age, as e.g. at an age 65+. However, the initiating age could be also extended to younger ages. In triggering all three steps, the system has the advantage for long-term care receivers to receive a pre-agreed annual income with optional escalation to help fund their care needs. Further, the invention has finally also the advantage to provide and trigger charges upfront for interest on a drawdown mortgage based on longevity. This enables homeowners to not sell a home nor run up significant interest debts (unknown) alongside it.

### BRIEF DESCRIPTION OF THE DRAWINGS

The present invention will be explained in more detail below relying on examples and with reference to these drawings in which:

FIG. 1 shows a block diagram, schematically illustrating a secured care equity advance multi-step trigger cycle 9 according to the inventive system. At the reference numeral 91, the system 1 is setting up care equity advance linkage, i.e. relationship. The reference numeral 92 denotes the process of the equity splitter, setting-up an equity split threshold splitting the equity in a care equity ratio 921 having a market value 9211 at the time point of establishing relationship (i.e. care equity advance linkage) 9212, and a cutoff equity ratio 922. 93 denotes the initializing of the care equity advance linkage, with 931 denoting the assigned up-front monetary amount, and 932 denoting the detection of the up-front monetary amount transfer. The reference numeral 94 denotes the triggering of a sufficient state of disability, and 95 denotes the draw-down monetary transfer up to equity split threshold with 951 in case of a periodic monetary transfer. It is to be noted that, in respect to the triggering or detecting of the sufficient state of disability, in the immediate need embodiment variant, the sufficient state of disability must be occurred, i.e. verifiable and/or detectable, before the up-front monetary amount. 96 denotes the start of dissolving care equity advance risk transfer by triggering the death of individual 41, 42, . . . , 4i or the sale of the asset 31, 32, . . . , 3i. Finally, the reference numeral 97 denotes the disintegration of equity ratios, with 971 denoting the relief of the care equity ratio 921 to system 1, and 972 the relief of cutoff equity ratio 922 to the care receiver's 41, 42, . . . , 4i estate or inheritance.

FIG. 2 shows a block diagram, schematically illustrating an automated system 1 for long-term care risk mitigation associated with immediate care needs of risk-exposed individuals in case of becoming a long-term care-receiver. The system 1 providing a secured multi-step trigger cycle 9 for risk-exposed individuals 4 thereby mitigating possibly occurring long-term care needs by means of the automated system based on a sufficient state of disability 2. The state of disability 2 of the risk-exposed individual 4 being measurable by a definable set of risk-exposed individual measuring parameters 10, wherein individual measuring parameter values 411, 421, . . . , 4i1 are captured and monitored by the automated system 10.

FIG. 3 shows a block diagram, schematically illustrating an example of a long-term care risk mitigation processed by the system 1. In the example, an individual 41, 42, . . . , 4i at 85 reaches the point he/she needs care. The individual 41, 42, . . . , 4i exemplarily has a £425k house 31, 32, . . . , 3i at a certain location. The individual 41, 42, . . . , 4i has an exemplary occupational pension income of £14.8 k pa (escalating at 3%)+state benefits. The individual 41, 42, . . . , 4i wants to plan for his/her care and for his/her to remain in her home for as long as possible. The individual 41, 42, . . . , 4i is prepared to use home equity if necessary. He/she wants to maximise the family's inheritance. The individual applies for long-term care risk mitigation (CEA) at the system 1 with £29.2 k of his/her savings. This provides a guaranteed interest-free annual benefit of £21.5 k pa, escalating at 3% pa. As FIG. 4 shows, the care equity ratio 921 is set to 50% of the asset 3 (i.e., home) value (£425 k). The individual's income has nearly doubled, due to the long-term care risk mitigation. The individual's net income is now £47 k pa, including CEA. In the example, the income tax may be exemplarily estimated at £2 k payable on state pension and private pension after taxable allowance. It is to be noted that in this UK example the state pension and attendance allowance are typically benefits provided by the state to which a care receiver would be entitled. Occupational pension may be based on 50% for i.e., non-manual female worker aged 50-59 average salary, escalated at 3% pa to simulate index-linked occupational pension.

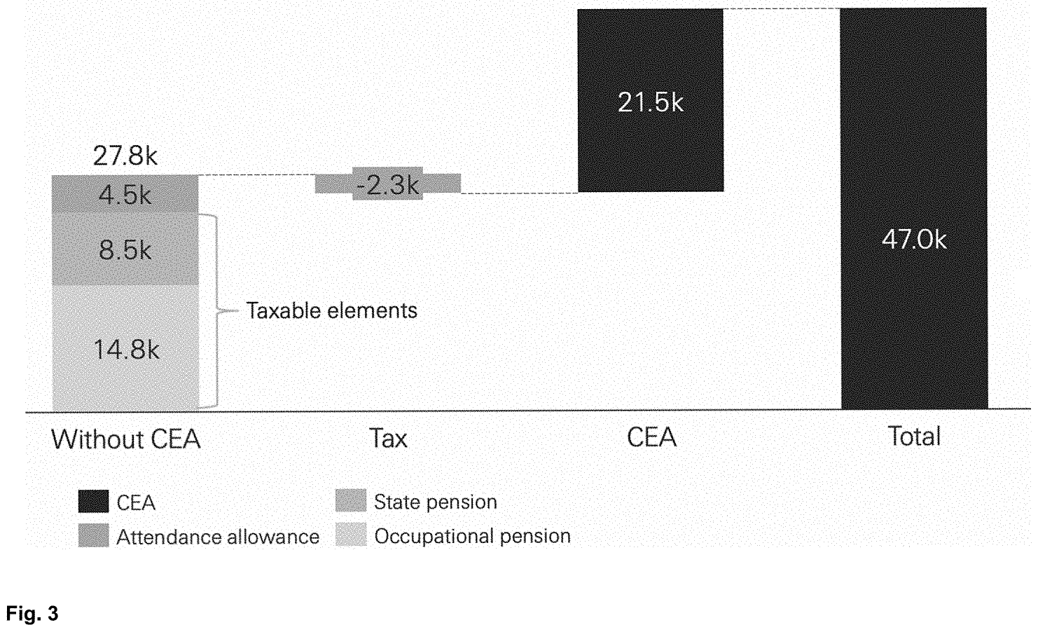

FIG. 4 shows a block diagram, schematically illustrating an example of risk and payment transfer functions occurring by applying the present system 1.

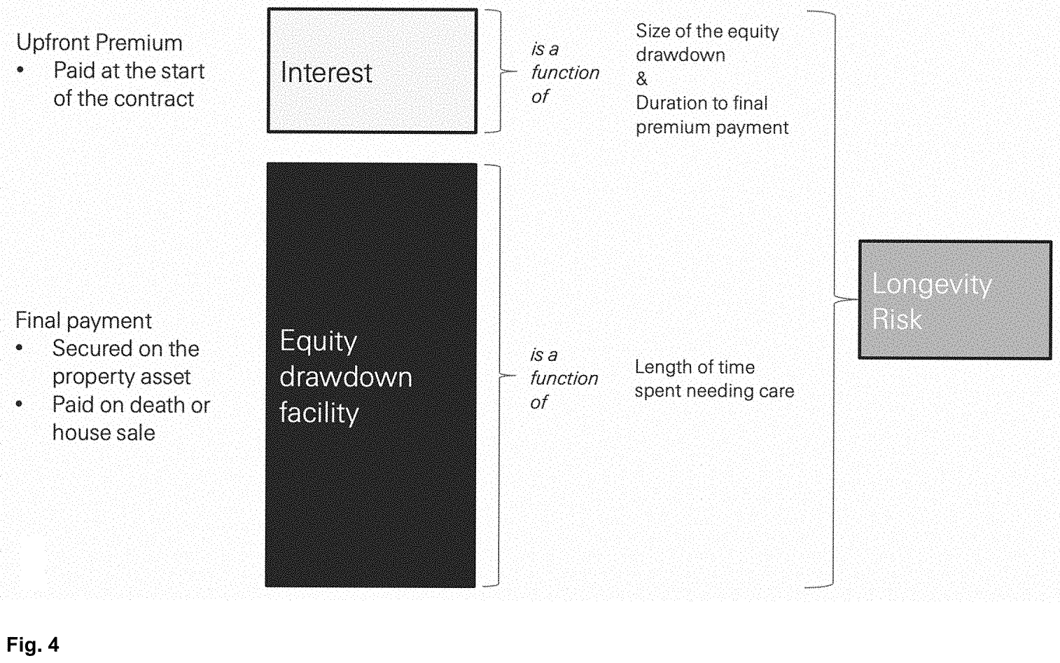

FIG. 5 shows a diagram, schematically illustrating systems' estimated loss ratios for different solutions. For the inventive long-term care risk mitigation system 1 (CEA) and immediate needs annuity system (INA) loss ratios worsen, i.e., become less profitable and subsequently loss-making, the longer the care receiver 41, 42, . . . , 4i lives. For lifetime mortgage system (LTM), the opposite is true—i.e., the mitigation becomes increasingly profitable the longer the care receiver 41, 42, . . . , 4i lives. FIG. 5 demonstrates how capabilities to understand, and price, longevity risk are essential components to CEA and INA but far less of a consideration for LTM.

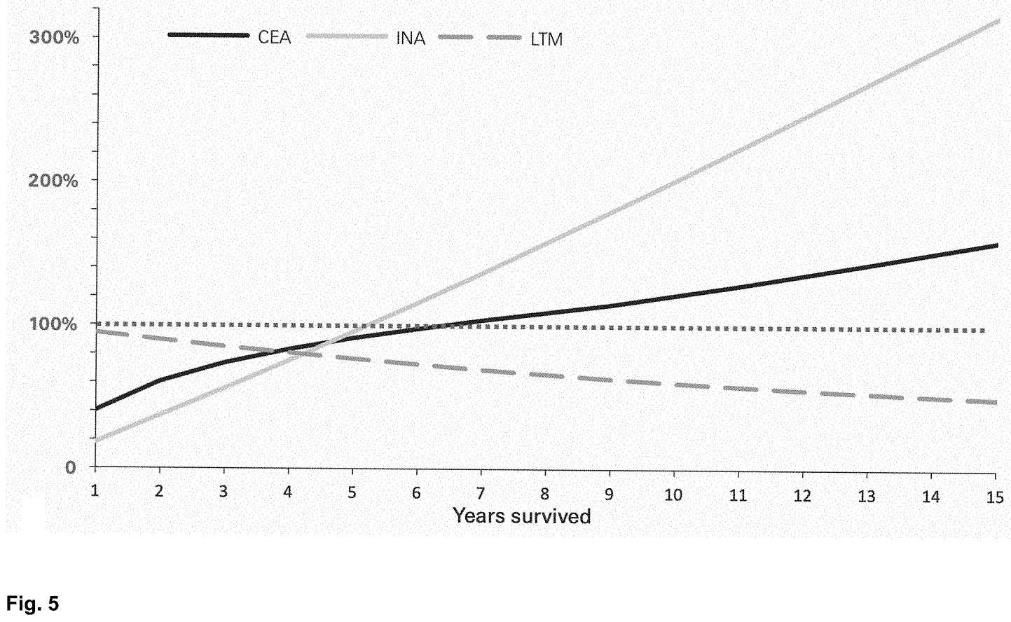

### DETAILED DESCRIPTION OF THE PREFERRED EMBODIMENTS

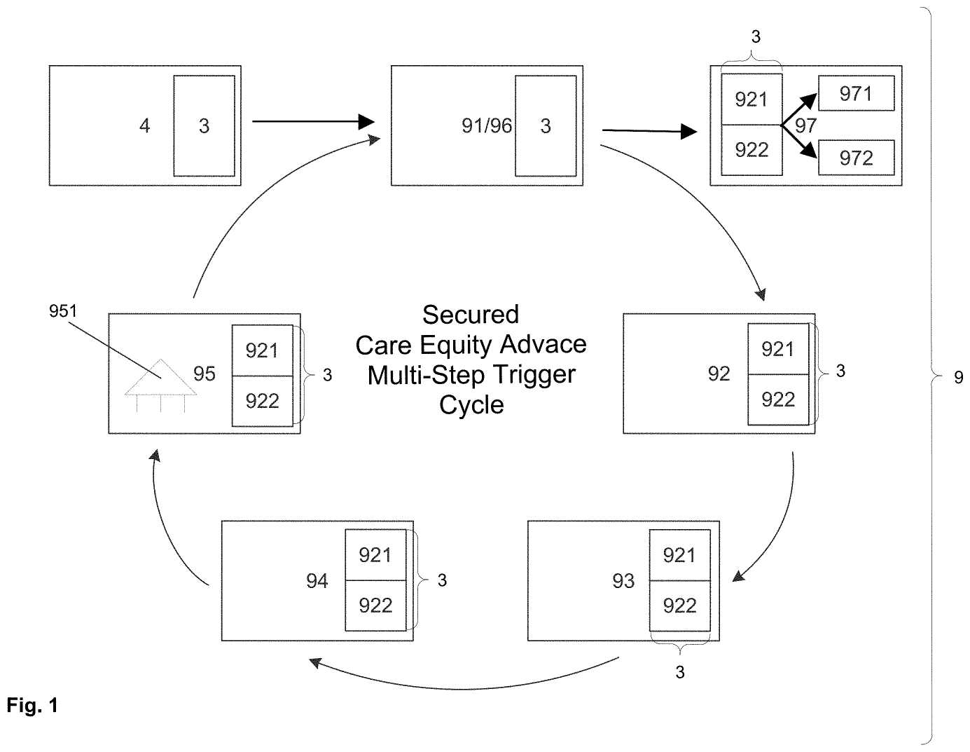

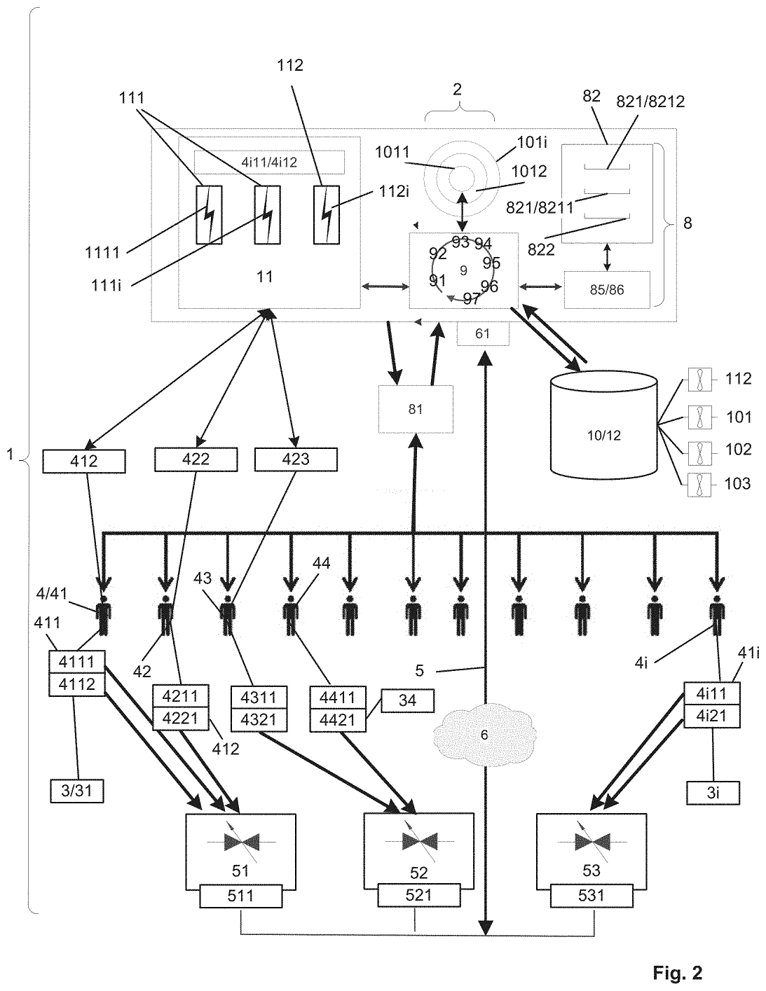

FIGS. 1 and 2 schematically illustrates an architecture for a possible implementation of an embodiment of an automated long-term care risk mitigation system 1 associated with immediate care needs of risk-exposed individuals in case of becoming a long-term care-receiver.

The system 1 provides a secured multi-step trigger cycle 9/91, 92, 93, 94, 95, 96, 97 for risk-exposed individuals 4 thereby mitigating possibly occurring long-term care needs by means of the automated system 1 based on a measured sufficient state of disability 2. The measured state of disability 2 of the risk-exposed individual 4 is monitored and/or detected using a definable set of risk-exposed individual measuring parameters 10, i.e. the individual measuring parameter values 411, 421, . . . , 4i1 being captured and monitored by the automated system 10.

The long-term care risk mitigation is triggered by means of an event-driven risk-exposed individual trigger 11 providing the secured multi-step trigger cycle 9. The event-driven risk-exposed individual trigger 11 comprising a staged multi-tier structure 111/112 gradually triggering threshold values 1111, . . . , 111i; 1121, . . . , 112i of a first and second set 101/102 of individual measuring parameters 10 in an autogenic dataflow pathway 5 of a risk-exposed individual 41, 42, 43, . . . , 4i. For each long-term care risk mitigation, long-term care mitigation data 121 are held in a persistence storage 12 of the system 1 comprising at least one assigned relationship 1211, 1212, . . . , 121i between an associated risk-exposed individual 41, 42, 43, . . . , 4i, an asset 3/31, 32, . . . , 3i of the risk-exposed individual 41, 42, 43, . . . , 4i and a remedial measure ratio 921 dependent on the asset 3/31, 32, . . . , 3i of the risk-exposed individual 41, 42, 43, . . . , 4i. The asset 3/31, 32, . . . , 3i assigned to the risk-exposed individual 41, 42, 43, . . . , 4i can e.g. be a real estate and/or the home of the risk-exposed individual 41, 42, 43, . . . , 4i. The remedial measures 822 can e.g. comprise transferred monetary remedial measures, wherein the transferal of the monetary remedial measures 822 is limited by the system 1 to a monetary amount determined by a predefined percentage 921 of the market value 9211 of the asset 31, 32, . . . , 3i. The market value 9211 of the asset 31, 32, . . . , 3i can e.g. be measured at the time point 9212 of establishing the assigned relationship 1211, 1212, . . . , 121i between the associated risk-exposed individual 41, 42, . . . , 4i and the asset 31, 32, . . . , 3i of the risk-exposed individual 41, 42, . . . , 4i. For example at the time point 9212 of underwriting the policy by the individual 41, 42, . . . , 4i. The predefined percentage 921 can e.g. amount to 50% to 80% of the market value 9211 of the asset 31, 32, . . . , 3i. However, it can also be chosen lower than 50%.

As a first option, in case of triggering a definable, sufficient state of disability 2 in combination with an age-related individual measuring parameter 1031 having a predefined threshold value 1032 amounting to a number of years, the assigned relationship 10i1 between the associated risk-exposed individual 31, 32, . . . , 3i and the asset 41, 42, . . . , 4i of the risk-exposed individual 31, 32, . . . , 3i can comprises an assigned up-front monetary amount 8211 generated by the system 1, wherein the long-term care risk mitigation is triggered by the system 1 upon detected transfer of the up-front monetary amount 8211 to the system 1. Thus, the relationship 1211, 1212, . . . , 121i providing the linkage for the long-term care risk mitigation is only established by the system 1 in the case that the defined up-front monetary amount 8211 is transferred to the system 1 and/or automated resource-pooling system 8. The predefined threshold value 1032 amounting to a number of years can e.g. be set to 65 years or more. It is to be noted, that the system allows to realize individualized underwriting and/or pricing. Whilst a specific embodiment variant of the invention can be based on detecting a minimum level of disability or cognitive decline as an “entry point”, where pricing will be relatively uniform other than differentiating by age, more advanced embodiment variant can include more individualized pricing and/or underwriting. As co-morbidities are common at older ages, life expectancy can vary greatly and just a few months' difference in expected longevity can move the required pricing of the product. Therefore, a more detailed and individualized underwriting and/or pricing procedure, which is closer to that followed by immediate needs annuities, can be realized by the system. This also involves more inputs for any underlying modelling based on medical history, diagnoses, capabilities to perform certain activities etc. Such a system provides a pricing specifically tailored for the individual policyholder.

As a second option, in that in case of triggering a healthy health status 1033 of the risk-exposed individual 41, 42, . . . , 4i based on the risk-exposed individual measuring parameter 10 in combination with an age-related individual measuring parameter 1031 having a predefined threshold value 1032 amounting to a number of years, the assigned relationship 10i1 between the associated risk-exposed individual 41, 42, . . . , 4i and the asset 31, 32, . . . , 3i of the risk-exposed individual 41, 42, . . . , 4i further comprises an assigned up-front monetary amount 8211 generated by the system 1, wherein the long-term care risk mitigation is triggered by the system 1 upon detected transfer of the up-front monetary amount 8211 to the system 1, and wherein the up-front monetary amount 8211 is either transferrable as up-front lump sum 82111 or as draw-down payment 82112 over a pre-defined time 82113. Also for the second option, the predefined threshold value 1032 amounting to a number of years can e.g. be set to 65 years or more. In the second option and in case of a draw-down payment transfer 82112, the resource-pooling system 8 can e.g. comprise a monitoring module 86 requesting a periodic payment transfer from the risk exposure individuals 41, 42, 43, . . . , 4i to the resource-pooling system 8 by means of a plurality of payment receiving modules 81, wherein the long-term care risk mitigation for the risk exposure components 41, 42, 43, . . . , 4x is interrupted by the monitoring module 86 when the periodic transfer is no longer detectable by means of the monitoring module 86 and/or the predefined assigned up-front monetary amount 8211 is not reached.

The staged multi-tier structure 111/112 comprises a first trigger stage 111 triggering 21 on a sufficient state of disability 2 of a selected risk-exposed individual 41, 42, 43, . . . , 4i by a first set of individual measuring parameter values 101 captured in the dataflow pathway 5 of the risk-exposed individual 41, 42, 43, . . . , 4i. The individual measuring parameter 101 being segmentable in critical categories of individual measuring data 101 at least comprising first trigger threshold parameter values 1111, 1112 . . . 111i providing a definable measure for individual's physical functional status parameters 1011 and/or current cognitive 1012 and/or sensory functional status parameters 1013 and/or prospective functional status parameters 1014 including physical and/or cognitive and/or sensory parameters 1015 and/or living environmental status parameters 1016 and/or long term care resource parameters 1017, the trigger threshold parameter values 1111, 1112, . . . 1117 triggered by means of monitored and measured values 411, 421, . . . , 4i1 of the first set of individual measuring parameters 101 in the autogenic dataflow pathway 5 of a risk-exposed individual 4. It is to be noted that the disclosed selection of measuring parameters cannot be contributed by a businessman with his abstract, closed business method nor can it be contributed within the frame of an abstract mathematical method, but necessarily come from the technical skilled person being familiar with the requirements of technical measurements, sensory systems and the requirements prediction modeling systems implemented on finite machines. It has further to be noted, that in the embodiment variant of the immediate needs, a disability state is needed before upfront monetary transfer. As mentioned, the long-term care risk mitigation is triggered by means of the event-driven patient-data trigger 11. The event-driven patient-data trigger 11 comprises a staged multi-tier structure 111/112 gradually triggering threshold values 1111, 1112, . . . , 1117/1121, . . . , 112i of the definable measuring parameters 10 in the autogenic dataflow pathway 5 of the risk-exposed individual 4. For example, the individual's dataflow pathway 5 can e.g. be monitored by the system 1 by capturing risk-related clinical and/or health-related measuring data 4i11 and/or assert-related data 4i12 in different dataflow pathways 5 associated with personal data of the risk-exposed individual 41, 42, . . . , 4i at least periodically and/or within predefined time frames. As such, the trigger threshold parameter values 1111, 1112, . . . 111i can be triggered by means of monitored and measured values 411, 421, . . . , 4i1 of the first set of individual measuring parameters 101 in the autogenic dataflow pathway 5 of a risk-exposed individual 41, 42, . . . , 4i departing from averaged historical parameter values and their value variations in time.

Upon detection 21/94 of the sufficient state of disability 2 based on the individual measuring parameters 101, the covering by means of remedial measures 822 is activated by signal transmission to a risk-cover structure 8 of the automated system 1 and by transferring the remedial measures 822 to the risk-exposed individual 41, 42, 43, . . . , 4i. The remedial measures 822 can comprise transferred monetary remedial measures, wherein the transferal of the monetary remedial measures is realized as periodic monetary remedial transfer to the risk-exposed individual 41, 42, . . . , 4i providing a predefined annual income or annuity to the to the risk-exposed individual 41, 42, . . . , 4i. The total amount of periodic monetary remedial measures transfer can e.g. be limited by the system 1 to a monetary amount determined by a predefined percentage of the market value 9211 of the asset 31, 32, . . . , 3i. The predefined percentage can e.g. amount to 50% to 80% of the market value 9211 of the asset 31, 32, . . . , 3i. As a variant, upon each detection 21 of the definable, sufficient state of disability 2 at the risk-exposed individual 41, 42, . . . , 4i, a total parametric payment as total of remedial measures 822 can be allocated with the triggering, wherein the transfer of a first portion of the totally allocated payment is triggered upon detection of the occurrence of a predefined up-front monetary amount 8211 within a predefined time window.

The multi-tier trigger structure 111/112 comprises a second stage trigger 112 triggering a second set of individual measuring parameter values 102 captured in the dataflow pathway 5 of the risk-exposed individual 41, 42, 43, . . . , 4i based on second trigger threshold parameter values 1121, 1122 . . . 112i. The second trigger threshold parameter values 1121, 1122 . . . 112i provide a definable measure for indicators for an occurred death of the risk-exposed individual and/or the sale of the real estate assigned to the risk-exposed individual 41, 42, 43, . . . , 4i and triggered by means of monitored and measured values 1021, 1022, . . . 102i of the measuring parameters 10 in the dataflow pathway 5 of the risk-exposed individual 41, 42, 43, . . . , 4i.

Upon detection 96 of the indicators for an occurred death of the risk-exposed individual and/or the sale of the asset assigned to the risk-exposed individual 41, 42, 43, . . . , 4i, the remedial measures 822 transferred by the risk-cover structure 8 of the system 1 are returned 823 to the system 1. The amount of remedial measures 822 being returned to the system 1 can e.g. be the total nominal value of the total remedial measures 921 transferred between the triggering of the first trigger stage 111 and the triggering of the second trigger stage 112. Further, upon detection of the indicators for an occurred death of the risk-exposed individual 41, 42, . . . , 4i and/or the sale of the asset 31, 32, . . . , 3i assigned to the risk-exposed individual 41, 42, . . . , 4i, a time trigger 851 can e.g. be initialized by the system 1 setting up a definable time window threshold 852 for returning the remedial measures 822 transferred by the risk-cover structure 8 to the system 1 and/or the automated resource-pooling system 8. The time window threshold 852 can e.g. be set to a size between 6 to 24 month. In particular, the time window threshold 852 can e.g. be set to 12 month.

FIG. 1 shows the secured care equity advance multi-step trigger cycle 9 according to the inventive system. The multi-step trigger cycle 9 comprises a first step cycle 91, where the system 1 is setting up care equity advance linkage, i.e. relationship. At a second step cycle 92, the process of the equity splitter 13 is setting-up an equity split threshold splitting the equity in a care equity ratio 921 having a market value 9211 at the time point of establishing relationship (i.e. care equity advance linkage) 9212, and a cutoff equity ratio 922. Thus, the cut off proportion is equal to the remaining amount (e.g. if the care equity ratio is, for example, 60% then the cut off ratio would be 40%). At a third step cycle 93, the care equity advance linkage is initialized, with reference numeral 931 denoting the assigned up-front monetary amount, and 932 denoting the detection of the up-front monetary amount transfer. At a forth step cycle 94, a sufficient state of disability is triggered and detected, respectively. Again, it is to be noted, that in the embodiment variant of the immediate needs, detecting or otherwise assuring the disability state is given before any possible upfront monetary transfer. At a fifth step cycle 95, the draw-down monetary is transferred by the system 1 and/or the automated resource pooling system 8 up to equity split threshold with the optional reference numeral 951 in case of a periodic monetary transfer. At a sixth step cycle 96, dissolving care equity advance risk transfer is initiated by triggering the death of individual 41, 42, . . . , 4i or the sale of the asset 31, 32, . . . , 3i. Finally, at a seventh step cycle 97, the equity ratios are disintegrated from the asset 31,32, . . . , 3i, where the reference numeral 971 denotes the relief of the care equity ratio 921 to system 1, and 972 the relief of cutoff equity ratio 922 to the care receivers 41, 42, . . . , 4i estate or inheritance.

In an embodiment variant, which is denoted as “immediate need embodiment variant” herein, detecting 21 or otherwise assuring the disability state occurs before any upfront monetary transfer. Thus, an upfront payment transfer can be requested by the system 1 either before or after detecting the sufficient state of disability, where the latter is the immediate need embodiment variant. In case of the system 1 being triggered by a definable, sufficient state of disability 2 in combination with an age-related individual measuring parameter 1031 having a predefined threshold value 1032 amounting to a number of years, the assigned relationship 10i1 between the associated risk-exposed individual 31, 32, . . . , 3i and the asset 41, 42, . . . , 4i of the risk-exposed individual 31, 32, . . . , 3i further requests an assigned up-front monetary amount 8211 generated by the system 1 after the occurring of a sufficient state of disability, wherein the long-term care risk mitigation is triggered by the system 1 upon detected transfer of the up-front monetary amount 8211 to the system 1.

In an embodiment variant, the detection 21 of the definable, sufficient state of disability 2 must be verified by an independent, associated verification and detection system based on the risk-exposed individual's measuring parameters 10, wherein the covering by means of remedial measures 211, 221, 231 is only activated upon additional verification confirmation of the independent verification and detection system. As a further embodiment variant, the second stage trigger 1112 can e.g. be dynamically adapted by means of an operating module 85 based on time-correlated incidence data indicative of a risk for attaining the definable, sufficient state of disability 2 by the risk-exposed individual 41, 42, . . . , 4i accounting for improvements in diagnosis or treatment and related risk measurements. As an even further embodiment variant, the system can comprise a indexation dependency. In a first variant, a pre-determined rate of inflation can e.g. be included (e.g. the amount the policyholder is entitled to would increase by 3% per year). As the cost of care tends to rise at a faster rate than headline inflation, this pre-determined amount can also be defined to be variable. In a more advanced variant, the system can provide a choice to the policyholder: the system would either be set at a specific percentage or related to an index, such as the local market's retail price index or another mechanism. This can allow the policyholder to combat inflation which can be particularly beneficial for those who live for a particularly long duration after subscribing to the policy.

It is to be noted, that the system's structure is further flexible. The system 1 can e.g. be realized to comprise an adaptability to evolving care needs. As an embodiment variant, the system 1 can be realized to be more or less rigid in the amount that the policyholder can draw down in any given year. However, the system can also e.g. comprise the inclusion of some “headroom” over and above the pre-defined amount required to fund care. As care needs can vary (e.g. if someone has a fall and breaks a bone, they might need to fund additional, more intensive care for a few weeks), other embodiment variant can be realized to have a more flexible structure, e.g. an automated allowance built in for additional care up to a specific limit each year that would be fully costed.

In the automated system 1 for long-term care mitigation associated with long-term care risks, the long-term care risk mitigation can e.g. be based on a signal transfer, in particular based on an electronic signal generation and transfer, to an associated, automated resource-pooling system 8 for risk sharing of long-term care risks of a variable number of risk exposure individuals 4/41, . . . , 4i by providing a dynamic self-sufficient risk protection for the risk exposure individuals 41, 42, 43, . . . , 4i by means of the resource-pooling system 8. The automated resource-pooling system 8 can be comprised in the system 1 or electronically associated steerable by electronic signal transfer. The risk exposure individuals 4/41, 42, 43, . . . , 4i can e.g. be connected to the resource-pooling system 8 by means of a plurality of payment-receiving modules 81 configured to receive and store 82 payments 821 from the risk exposure individuals 4/41, 42, 43, . . . , 4i. The resource-pooling system 8 can e.g. be activated by means of the event-driven patient-data trigger 11 comprising the staged multi-tier trigger structure 111/112 triggering in a patient dataflow pathway 5/412, 422, . . . , 4i2 to provide risk protection for a specific risk exposure individuals 4 based on received and stored payments 821 of the risk exposure individuals 4. In case an occurrence of long-term care needs in the patient data flow pathway 5 of a risk exposure individual 4 can e.g. be triggered or detected, a corresponding trigger-flag is set by means of the automated resource-pooling system 8 and a transfer of payments, in particular a parametric payment transfer, is assigned to this corresponding trigger-flag, wherein long-term care needs can be distinctly covered by the resource-pooling system 8 based on the respective trigger-flag and based on the received and stored payment parameters 821 from risk exposure individuals 4/41, 42, 43, . . . , 4i by means of the transfer from the resource-pooling system 8 to the risk exposure individual 4/41, 42, 43, . . . , 4i.

### LIST OF REFERENCE SIGNS

1 System for medical intervention risk mitigation
   10 Risk-exposed individual measuring parameters
      101 First set of individual measuring parameters
         1011 Individual's physical functional status parameter (1^{st} category)
         1012 Cognitive measuring parameters (2^{nd} category)
         1013 Sensory functional status parameters (3^{rd} category)
         1014 Prospective functional status parameters (4^{th} category)
         1015 Sensory parameters (5th category)
         1016 Living environmental status parameters (6th category)
         1017 Long term care resource parameters (7th category)
      102 Second set of individual measuring parameters
         1021, 1022, . . . , 102i Measuring parameters of second parameter set
      103 Third set of individual measuring parameters
         1031 Age-related individual measuring parameter
         1032 Age-related threshold value
         1033 Health status parameter
   11 Event-driven risk-exposed individual trigger
      111 First stage trigger triggering on sufficient state of disability
         1111 Trigger threshold value of physical functional status parameter
         1112 Trigger threshold value of cognitive measuring parameters
         1113 Trigger threshold value of sensory functional status parameters
         1114 Trigger threshold value of prospective functional status parameters
         1115 Trigger threshold value of sensory parameters
         1116 Trigger threshold value of living environmental status parameters
         1117 Trigger threshold value of long term care resource parameters
      112 Second stage equity care advance trigger
         1121 Trigger threshold value of parameter 1021
         1122 Trigger threshold value of parameter 1022
         . . .
         112i Trigger threshold value of parameter 102i
   12 Persistence storage
      121 Long-term care mitigation data
         1211, 1212, . . . , 121i Care equity advance relationship assigned to individual i
         121i1 Risk-exposed individual identification of individual i
         121i2 Asset identification assigned to individual i
         121i21 Location of asset
         121i22 Market value of asset
   13 Equity splitter
2 Sufficient state of disability
   21 Triggering for sufficient state of disability
   22 Detection of occurring long-term care needs
3 Asset/Real estate
   31, 32, . . . , 3i Asset assigned to risk-exposed individual i
4 Risk-exposed individual/potential care receiver in a secured care equity advance multi-step cycle
   41, 42, 43, . . . , 4i Selected risk-exposed individual
      411, 421, . . . , 4i1 Individual measuring parameter values
         4i11 Clinical and/or health related measuring parameters
         4i111 Health related measuring parameters
         4i112 Occurrence of death related measuring parameters
         4i12 Asset-related measuring parameters
      412, 422, . . . , 4i2 Specific patient dataflow pathway of individual 4x
      413, 423, . . . , 4i3 Stored payment parameters associated with an individual
5 Autogenic dataflow pathway of the risk-exposed individual
   51 Sensors
      511 Data transmission interface of sensors 51
   52 Measuring devices
      521 Data transmission interface of measuring devices 52
   53 Data input devices
      531 Data transmission interface of data input devices 53
6 Data transmission network
   61 Data transmission interface
7 Splitter
8 Automated resource pooling system
   81 Payment-receiving modules
   82 Payment data store
      821 Stored payment parameters associated with individual's monetary Transfer
         8211 Definable up-front monetary amount parameters
         82111 Up-front monetary amount parameters
         82112 Draw-down payment transfer parameters for up-front monetary amount
         82113 Time threshold for draw-down payment transfer
         8212 Care equity ratio resolve parameters
      822 Stored parameters denoting the available remedial measures
         8223 Threshold value for remedial measures as percentage of asset value
   84 Assembly module
   85 Operating module
      851 Time trigger
      852 Time window threshold
   86 Monitoring module
9 Secured multi-step trigger cycle engine
   91 Setting up care equity advance linkage
   92 Equity splitter: set-up of equity split threshold
      921 Care equity ratio
         9211 Market value
         9212 Time point of establishing relationship (care equity advance linkage)
      922 Cutoff equity ratio
   93 Initializing care equity advance linkage
      931 Assigned up-front monetary amount
      932 Detecting up-front monetary amount transfer
   94 Triggering sufficient state of disability
   95 Draw-down monetary transfer up to equity split threshold
      951 Periodic monetary transfer
   96 Dissolving care equity advance mitigation/Triggering death of individual
   97 Disintegration of equity ratios
      971 Relief of care equity ratio to system
      972 Relief of cutoff equity ratio to care receiver estate

1. An automated, event-driven, and triggered system for long-term care risk mitigation associated with detected and/or measured immediate care needs of risk-exposed individuals in case of becoming a long-term care-receiver, the automated system providing a secured multi-step trigger and/or detection cycle for a risk-exposed individual based on measured sensory measuring data thereby mitigating possibly occurring long-term care needs by means of the automated system based on a sufficient state of disability of the risk-exposed individual, the sufficient state of disability being characterized by a definable set of risk-exposed individual measuring parameters, and individual measuring parameter values being captured and monitored by the automated system, the automated system being configured to:
monitor a dataflow pathway of the risk-exposed individual by capturing risk-related clinical and/or health-related measuring data and asset-related data in different dataflow pathways associated with personal data of the risk-exposed individual periodically or within predefined time frames, and
trigger the long-term care risk mitigation by means of an event-driven risk-exposed individual trigger providing the secured multi-step trigger cycle, wherein
the event-driven risk-exposed individual trigger includes a staged multi-tier structure gradually triggering threshold values of a first and second set of individual measuring parameters in an autogenic dataflow pathway of the risk-exposed individual,
for each long-term care risk mitigation, long-term care mitigation data are held in a persistence storage of the automated system and include at least one assigned relationship between the risk-exposed individual, an asset of the risk-exposed individual, and a remedial measure ratio dependent on the asset of the risk-exposed individual,
the staged multi-tier structure includes a first trigger stage triggering on the sufficient state of disability of the risk-exposed individual by a first set of the individual measuring parameter values captured in the dataflow pathway of the risk-exposed individual,
the first set of the individual measuring parameter values are segmentable in critical categories of individual measuring data at least including first trigger threshold parameter values providing a definable measure for individual physical functional status parameters and/or current cognitive and/or sensory functional status parameters and/or prospective functional status parameters including physical and/or cognitive and/or sensory parameters and/or living environmental status parameters and/or long term care resource parameters,
the first trigger threshold parameter values are triggered by means of monitored and measured values of the first set of the individual measuring parameter values in the autogenic dataflow pathway of the risk-exposed individual departing from averaged historical parameter values and their value variations in time,
upon detection of the sufficient state of disability based on the first set of the individual measuring parameter values, covering by means of remedial measures is activated by electronic signal transmission to an automated risk-cover structure of the automated system being steerable by the electronic signal transmission and by transferring the remedial measures to the risk-exposed individual,
the multi-tier trigger structure includes a second stage trigger triggering a second set of the individual measuring parameter values captured in the dataflow pathway of the risk-exposed individual based on second trigger threshold parameter values providing a definable measure for indicators for an occurred death of the risk-exposed individual and/or a sale of the asset assigned to the risk-exposed individual,
the second threshold parameter values are triggered by means of monitored and measured values of the second set of the individual measuring parameter values in the autogenic dataflow pathway of the risk-exposed individual,
the second stage trigger is dynamically adapted by means of an operating module based on time-correlated incidence data indicative of a risk for attaining the sufficient state of disability by the risk-exposed individual accounting for improvements in diagnosis or treatment and related risk measurements,
upon detection of the indicators for the occurred death of the risk-exposed individual and/or the sale of the asset assigned to the risk-exposed individual, the remedial measures transferred by the automated risk-cover structure of the automated system are returned to the automated system, and
an amount of the remedial measures being returned to the automated system is a total nominal value of total remedial measures transferred between the triggering of the first trigger stage and the triggering of the second trigger stage.

2. The automated system according to claim 1, wherein the asset assigned to the risk-exposed individual is a real estate of the risk-exposed individual.

3. The automated system according to claim 1, wherein
the remedial measures include transferred monetary remedial measures, and
transferal of the monetary remedial measures is limited to a monetary amount determined by a predefined percentage of a market value of the asset.

4. The automated system according to claim 3, wherein the market value of the asset is measured at a time point of establishing the assigned relationship between the risk-exposed individual and the asset of the risk-exposed individual.

5. The automated system according to claim 3, wherein the market value of the asset is measured or reevaluated at a time point of detecting or triggering the sufficient state of disability.

6. The automated system according to claim 3, wherein the predefined percentage amounts to equal or less than 50% to 80% of the market value of the asset.

7. The automated system according to claim 1, wherein upon the detection of the indicators for the occurred death of the risk-exposed individual and/or the sale of the asset assigned to the risk-exposed individual, a time trigger is initialized by setting up a definable time window threshold for returning the remedial measures transferred by the automated risk-cover structure to the automated system.

8. The automated system according to claim 7, wherein the time window threshold is set to a size between 6 to 24 months.

9. The automated system according to claim 8, wherein the time window threshold is set to 12 months.

10. The automated system according claim 1, wherein
in case of triggering the sufficient state of disability in combination with an age-related individual measuring parameter having a predefined threshold value amounting to a number of years, the at least one assigned relationship between the risk-exposed individual and the asset of the risk-exposed individual further comprises an up-front monetary amount generated by the automated system, and
the long-term care risk mitigation is triggered by the automated system upon detected transfer of the up-front monetary amount to the automated system.

11. The automated system according to claim 1, wherein
in case of triggering a healthy health status of the risk-exposed individual based on the risk-exposed individual measuring parameter in combination with an age-related individual measuring parameter having a predefined threshold value amounting to a number of years, the at least one assigned relationship between the risk-exposed individual and the asset of the risk-exposed individual further comprises an up-front monetary amount generated by the automated system,
the long-term care risk mitigation is triggered by the automated system upon detected transfer of the up-front monetary amount to the automated system, and
the up-front monetary amount is either transferrable as an up-front monetary amount or as a draw-down payment transfer over a pre-defined time.

12. The automated system according to claim 10, wherein the predefined threshold value amounting to the number of years is set to 65 years or more.

13. The automated system according to claim 11, wherein
in case of the draw-down payment transfer, the automated risk-cover structure includes a monitoring module requesting a periodic payment transfer from the risk-exposed individual to the automated risk-cover structure by means of a plurality of payment receiving modules, and
the long-term care risk mitigation is interrupted by the monitoring module when the periodic payment transfer is no longer detectable by means of the monitoring module and/or the up-front monetary amount is not reached.

14. The automated system according to claim 1, wherein
the remedial measures include transferred monetary remedial measures, and
transferal of the monetary remedial measures is realized as periodic monetary remedial transfer to the risk-exposed individual providing a predefined annual income or annuity to the to the risk-exposed individual.

15. The automated system according to claim 14, wherein a total amount of the periodic monetary remedial transfer is limited by the automated system to a monetary amount determined by a predefined percentage of a market value of the asset.

16. The automated system according to claim 15, wherein the predefined percentage amounts to 50% to 80% of the market value of the asset.

17. The automated system according to claim 1, wherein
detection of the sufficient state of disability is verified by an independent verification and detection system based on the risk-exposed individual measuring parameters, and
the covering by means of the remedial measures is only activated upon additional verification confirmation of the independent verification and detection system.

18. The automated system according to claim 1, wherein
upon each detection of the sufficient state of disability of the risk-exposed individual, a total parametric payment as total of the remedial measures is allocated with the triggering, and
the transfer of a first portion of the total parametric payment is triggered upon detection of occurrence of a predefined up-front monetary amount within a predefined time window.
